(19) United States
(12) Patent Application Publication
(10) Pub. No.: US 2022/0298515 A1
(43) Pub. Date: Sep. 22, 2022

(54) ULTRASOUND-BASED THERMOGENETICS FOR IMMUNOTHERAPY

(71) Applicant: The Regents of the University of California, Oakland, CA (US)

(72) Inventors: Yingxiao WANG, San Diego, CA (US); Shu CHIEN, San Diego, CA (US); Yiqian WU, San Diego, CA (US); Shaoying LU, San Diego, CA (US)

(21) Appl. No.: 17/642,186

(22) PCT Filed: Sep. 10, 2020
(86) PCT No.: PCT/US2020/050094

§ 371 Date: Mar. 10, 2022

Related U.S. Application Data
(60) Provisional application No. 62/898,280, filed on Sep. 10, 2019.

(51) Int. Cl. C12N 15/63; C07K 14/725; C07K 14/47; A61P 35/00

(52) U.S. Cl. CPC C12N 15/635; C07K 14/7051; C07K 14/47; A61P 35/00; C07K 2319/03; A61K 38/00

(57) ABSTRACT

Provided are compositions, products of manufacture, kits and methods for remotely-controlled and non-invasive manipulation of intracellular nucleic acid expression, genetic processes, function and activity in live cells such as T cells in vivo, for activating, adding functions or changing or adding specificities for immune cells, for monitoring physiologic processes, for the correction of pathological processes and for the control of therapeutic outcomes. Provided are ultrasound-based stimulations and a heat-sensitive activation of proteins caused by the activation of polypeptides controlled by heat shock protein promoters to control the production of intracellular nucleic acid and gene expression, for example, for the expression of biological-active proteins such as T cell receptors, for example, chimeric antigen receptors (CARs). Thermogenetic systems as provided herein can be based on ultrasound and/or heat, allowing a deep penetration of stimulation and manipulation in vivo at centimeter-level depth with high spatiotemporal precision.

### RELATED APPLICATIONS

This Patent Convention Treaty (PCT) International Application claims the benefit of priority to U.S. Provisional Application Ser. No. (USSN) 62/898,280 filed Sep. 10, 2019. The aforementioned application is expressly incorporated herein by reference in its entirety and for all purposes.

### STATEMENT AS TO FEDERALLY SPONSORED RESEARCH

This invention was made with government support under GM126016, GM125379, and HL121365 awarded by the National Institutes of Health. The government has certain rights in the invention.

### TECHNICAL FIELD

This invention generally relates to thermogenetics, cell biology and ultrasound technologies. In alternative embodiments, provided are compositions, including products of manufacture and kits, and methods, for remotely-controlled and non-invasive manipulation of intracellular nucleic acid expression, genetic processes, function and activity in live cells such as a T cell, a primary T cell, a B cell, a monocyte, a macrophage, a dendritic cell or a natural killercell in vivo, for example, activating, adding functions or changing or adding specificities for immune cells, for monitoring physiologic processes, for the correction of pathological processes and for the control of therapeutic outcomes. In alternative embodiments, provided are ultrasound-based stimulations and a heat-sensitive activation of proteins caused by the activation of polypeptides controlled by heat shock protein promoters to control the production of intracellular nucleic acid and gene expression, for example, for the expression of biological-active proteins such as T cell or NK cell receptors, for example, chimeric antigen receptors (CARs), which can be used, in alternative embodiments, for diagnostic or therapeutic purposes. In alternative embodiments, exemplary thermogenetic systems are provided herein, being based on ultrasound and/or heat, allowing a deep penetration of stimulation and manipulation in vivo at centimeter-level depth with high spatiotemporal precision.

### BACKGROUND

Artificial T cell receptors (also known as chimeric T cell receptors, chimeric immunoreceptors, chimeric antigen receptors (CARs)) are engineered receptors, which graft a desired specificity onto an immune effector cell such as a T cell. CAR T cell therapy is becoming a paradigm-shifting therapeutic approach for cancer treatment, particularly with the benefit of resulted central memory T cells capable of lasting for months to years in suppressing the cancer relapse. In this therapy, T cells are removed from a cancer patient and modified to express CARs that target the cancer. These modified T cells, which can recognize and kill the patient's cancer cells, are re-introduced into the patient.

However, major challenges remain before CAR-based immunotherapy can become widely adopted. For instance, the non-specific targeting of the CAR-T cells against normal/nonmalignant tissues (on-target but off-tumor toxicities) can be life-threatening. In fact, off-tumor toxicities against the lung, gray matter in the brain, and cardiac muscles, have caused multiple cases of deaths. While synthetic biology and genetic circuits have been used in attempts to address this issue, there is an urgent need for high-precision control of CAR-T cells to confine the activation in tissue space.

In immunotherapy, the expression of engineered CAR on the cell surface enables T cells to recognize specific antigens on the target cell. This triggers T cell activation and can eventually lead to the elimination of target cells. Clinical trials involving anti-CD19 CAR T cells against B-cell malignancies have shown promising results, demonstrating the therapeutic effects of CAR T cells in cancer treatment. However, the perfusion of constitutively activated CAR T cells into patients may have lethal consequences due to the induced cytokine storm and ‘on-target, off tumor’ toxicity. Therefore, researchers are actively seeking control over the timing and location of the activation of the perfused CAR T cells. Given the complexity of immune system and the largely overlapping functions of its molecular regulators, it is a daunting challenge to manipulate immune system at global levels with predictable net outcomes.

### SUMMARY

In alternative embodiments, provided are methods for remotely-controlling and non-invasively manipulating expression of an exogenous nucleic acid in a cell, or an immune cell, and optionally modifying or adding a target capability or a function to the cell, or immune cell,

wherein optionally the immune cell is a T cell, a primary T cell, a B cell, a monocyte, a macrophage, a dendritic cell or a natural killercell,

wherein optionally the exogenous nucleic acid is contained in a vector or expression cassette,

and optionally the exogenous nucleic acid comprises a nucleic acid encoding (expressing) a protein, and optionally the protein is a therapeutic protein, or a transcriptional or translational regulatory protein, or a receptor, or a recombinant or an artificial T cell or NK cell receptor (also known as a chimeric T cell or NK receptor, a chimeric immunoreceptor, a chimeric antigen receptor and a CAR), an antibody, a single chain antibody, or a single-domain antibody (also known as sdAb or nanobody) or an antibody fragment consisting of a single monomeric variable antibody domain,

the method comprising:

(a) inserting or expressing in a cell, an immune cell or a plurality of cells or immune cells, a thermo-responsive nucleic acid, wherein the thermo-responsive nucleic acid comprises the exogenous nucleic acid operatively linked to a thermo-responsive promoter (or a mammalian or a human promoter or transcriptional activator activated by increased temperature) to form a Gene Transducing Module (GTM), wherein optionally the thermo-responsive promoter is or comprises a heat shock protein (Hsp) promoter or equivalent, optionally a human heat shock protein 70B (Hsp) promoter,

and optionally the human heat shock protein 70B (Hsp) promoter has a sequence as set forth in SEQ ID NO:1:

(SEQ ID NO: 1)
GTCGAGGCGCGTCCTCAGAGCCAGCCGGGAGGAGCTAGAACCTTCCCCGC
GTTTCTTTCAGCAGCCCTGAGTCAGAGGCGGGCTGGCCTGGCATAGCCGC
CCAGCCTCTCGGCTCACGGCCCGATCCGCCCGAACCTTCTCCCGGGGTCA
GCGCCGCGCTGCGCCGCCCGGCTGACTCAGCCCGGGCGGGCGGGCGGGA
GGCTCTCGACTGGGCGGGAAGGTGCGGGAAGGTTCGCGGCGGCGGGGTC
GGGGAGGTGCAAAAGGATGAAAAGCCCGTGGAAGCGGAGCTGAGCAGAT
CCGAGCCGGGCTGGCGGCAGAGAAACCGCAGGGAGAGCCTCACTGCTGA
GCGCCCCTCGACGGCGGAGCGGCAGCAGCCTCCGTGGCCTCCAGCATCCG
ACAAGAAGCTCTCTAGTCGACGGTATCGAT;

(b) stimulating or exposing the cell to a heat or a heat source sufficient to cause the thermo-responsive promoter to be activated, thereby causing the thermoresponse protein to be expressed in the cell.

In alternative embodiments of methods as provided herein:
the expressing of the thermoresponse protein in the cell adds a function to the cell, or immune cell, or manipulates a physiologic and/or a genetic process in the cell, or immune cell, and optionally when the upregulated nucleic acid is a nucleic acid expressing (encoding) a CAR, a single chain antibody, or a single-domain antibody (also known as sdAb or nanobody) or an antibody fragment consisting of a single monomeric variable antibody domain, thereby adding a new specificity, function or target cell to a cell, an immune cell or a T cell;
the cell is a human cell or a mammalian cell, or is a recombinantly engineered cell engineered to contain or comprise the Gene Transducing Module (GTM), transplanted or inserted into a tissue, an organ, an organism or an individual, or is a non-human transgenic animal genetically engineered to contain and express the Gene Transducing Module (GTM) or vector;
the cell is exposed to a high-intensity focused ultrasound (HIFU) and/or focused ultrasound (FUS), thereby generating sufficient heat in the cell to cause the thermo-responsive promoter to be activated, thereby causing the thermoresponse protein to be expressed in the cell, and optionally the cell is heated to between about 40° C. and 48° C., or to between about 42° C. and 45° C., or to about 43° C.;
the cell is inside the body of an animal or a human in need thereof, and the HIFU and/or FUS is focused on or approximate to a tumor, a cancer or a dysplastic or dysfunctional tissue; and/or
the method is used for the manipulation or correction of a pathological process, optionally, for eradicating a tumor or a cancer in an individual in vivo, wherein optionally the individual is a human or an animal.

In alternative embodiments, provided are uses of a genetically engineered cell as engineered for uses or methods as provided herein, as a medicament.

In alternative embodiments, provided are uses of a genetically engineered cell as engineered for uses or methods as provided herein, as a medicament in a remotely-controlled and non-invasive manipulation of a physiologic and/or a genetic process in a cell, or an immune cell, or for the addition of a function or a target specificity to the cell, or immune cell, or plurality of cells or immune cells, or for the manipulation or correction of a pathological process, optionally, for eradicating a tumor or a cancer in an individual in vivo.

In alternative embodiments, provided are genetically engineered cells as engineered for uses or methods as provided herein for use as a medicament, or for use as a medicament in a remotely-controlled and non-invasive manipulation of a physiologic and/or a genetic process in a cell, or an immune cell, or for the addition of a function or a target specificity to the cell, or immune cell, or plurality of cells or immune cells, or for the manipulation or correction of a pathological process, optionally, for eradicating a tumor or a cancer in an individual in vivo.

Given the complexity of immune system and the largely overlapping functions of its molecular regulators, it is a daunting challenge to manipulate immune system at global levels with predictable net outcomes, and embodiments as provided herein address this problem, and as such, the capability of the methods as provided herein in controlling immune activities at local regions with high precision in vivo will have broad impact and usage for immune engineering and therapeutics.

The details of one or more exemplary embodiments as described herein are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 schematically illustrates the remote activation of the production of biologically active molecules (for example, anti-CD19 chimeric antigen receptor (CAR)) by ultrasound-induced heat shock. The T cell is engineered to carry an exemplary Gene Transducing Module (GTM) as provided herein, which can comprise a heat shock protein (Hsp) promoter and an anti-CD19 CAR. In alternative embodiments, high-intensity focused ultrasound (HIFU) and/or focused ultrasound (FUS) is applied to generate heat at the location of the engineered T cells. Upon ultrasound-induced heat shock, the heat shock factor (HSF) monomers in the cytoplasm form homotrimers and translocate into the nucleus, where they bind to the Hsp promoter and activate downstream gene expression. The biologically active CAR, for example, anti-CD19 CAR, is then synthesized and expressed on the cell surface, where it recognizes the antigen, for example, the CD19 antigen, expressed on the surface of the tumor or the cancer cell and triggers the killing of the tumor or the cancer cell.

FIG. 2A-B illustrates heat induced gene expression in HEK cells:

FIG. 2A schematically illustrates an exemplary Gene Transducing Module (GTM) design in the upper panel, where a heat shock protein (Hsp) promoter driving the reporter eGFP was used as the heat-sensitive GTM with a constitutively expressing mCherry serving as a normalization reference to minimize cell-cell heterogeneity; and the images on the lower panels graphically illustrate the heat-induced gene expression of the GTM, where the left image shows the reporter gene expression in heat shock or control groups; the middle image shows the constitutive reference mCherry expression; and the right image shows Differential Interference Contrast (DIC) images of the cells; and

FIG. 2B on the upper panel schematically illustrates the exemplary GTM contains an Hsp promoter driving the anti-CD19 CAR and the reporter eGFP; and the images on the lower panels show the heat-induced gene expression of the GTM, where the left image shows the reporter gene expression in heat shock or control groups; and the right image shows Differential Interference Contrast (DIC) images of the cells.

FIG. 3A-D illustrates the expression of genes of interest in Jurkat T cells carrying the designed exemplary Gene Transducing Modules (GTMs) after heat shock induction:

FIG. 3A lower images illustrate representative flow cytometry data showing the expression of eGFP in Jurkat cells transduced with the Hsp promoter driven e-green fluorescent protein (GFP) (eGFP) 13 hours (hr) after heat shock; where the left image shows the control group, and the right image shows the heat shock group, and the FIG. 3A upper image illustrates the exemplary GTM;

FIG. 3B illustrates representative images showing the expression of eGFP 18 hr after heat shock in Jurkat cells transduced with the GTM in FIG. 3A, with lower panels showing Differential Interference Contrast (DIC) images of control (left image) and heat shock (right image);

FIG. 3C lower panels graphically illustrate representative flow cytometry data of Jurkat cells expressing the eGFP tagged anti-CD19 CAR driven by the Hsp promoter 13 hr after heat shock, with the left panel showing the control group, and the right panel showing the heat shock group, and the upper panel showing the exemplary GTM; and

FIG. 3D illustrates representative images showing the expression of eGFP 23 hr after heat shock in Jurkat cells transduced with the exemplary GTM illustrated in FIG. 3C, with lower panels showing Differential Interference Contrast (DIC) images of control (left image) and heat shock (right image).

FIG. 4 graphically illustrates CD69 expression as an indicator of Jurkat activation upon interacting with Toledo cells: CD69 expression was measured in untransfected Jurkats (yellow), Jurkats hosting a Gene Transducing Module (GTM) with (red) or without heat shock (blue). This GTM is composed of an Hsp promoter, an anti-CD19 CAR and eGFP. The Jurkat cells 12 hr after heat shock stimulation were mixed with Toledo cells. CD69 staining was performed after another 24 hr using a fluorophore-conjugated anti-CD69 antibody and analyzed by flow cytometry. In all cases, heat shock was conducted by incubating the cells in 43° C. incubator for 60 min, while the control group was incubated at 37° C.

FIG. 5 graphically illustrates heat shock induced gene expression in primary human T cells: Primary human T cells were introduced with the heat-sensitive GTM containing the Hsp promoter-driven e-green fluorescent protein (GFP) (eGFP) with a constitutive mCherry. The induction of eGFP was quantified in terms of percentage and mean intensity quantified from flow cytometry data in the mCherry+ gate. Different heat shock patterns were tested using a thermal cycler. Heat shock patterns “(ON, OFF) min×number of cycles” represent that the cells were kept at ON (43° C.) and OFF (37° C.) statuses for the indicated durations, and this cycle was repeated for the indicated number of times.

FIG. 6A-C illustrates heat-inducible CAR expression and cytotoxicity in primary human T cells:

FIG. 6A schematically illustrates exemplary GTMs used in this study;

FIG. 6B graphically illustrates representative flow cytometry data showing CD19CAR expression in engineered primary human T cells without (CT) or with (HS) 43° C. 15 min heat shock as quantified by antibody staining; and

FIG. 6C graphically illustrates cytotoxicity of the T cells in FIG. 6B against Fluc+ Nalm-6 tumor cells (a B cell precursor leukemia cell line) at varying E:T ratios.

FIG. 7A-D illustrate remote activation of gene in vivo using Magnetic Resonance Imaging (MRI)-guided focused ultrasound (FUS):

FIG. 7A schematically illustrates an exemplary MRI-guided focused ultrasound (FUS) system used in this study;

FIG. 7B illustrates Magnetic Resonance (MR) images showing coronal and axial views of the ultrasound transducer and the coronal view of a mouse under focused ultrasound (FUS) stimulation with color-coded temperature map superimposed; white arrow in lower left image points to the focus of the ultrasound;

FIG. 7C graphically illustrates the activation of reporter Fluc gene by FUS stimulation in vivo quantified by the ratio of Fluc and Rluc intensities: HIFU−: mice without FUS stimulation; HIFU+: mice with 2×5 min FUS stimulation at 43° C.;

FIG. 7D illustrates representative IVIS™ (combines 2-dimensional (2D) optical and 3-dimensional (3D) optical tomography in one platform) (Perkin Elmer) in vivo images showing the Fluc intensities in a mouse before and after FUS stimulation; *: p<0.05 by Two-way ANOVA with Tukey's multiple comparisons test.

FIG. 8A-E illustrates the focused ultrasound (FUS)-controllable tumor suppression by the engineered CAR T cells in vivo:

FIG. 8A schematically illustrates the timeline (right image) of the in vivo experiment using NSG (NOD scid gamma mouse, Jackson Laboratories) mouse bearing matched bilateral tumors (left image) as the animal model; the tumor on the left flank received focused ultrasound (FUS) stimulation (FUS+) and the one on the right received no FUS (FUS−) following injection of engineered CAR T cells;

FIG. 8B and FIG. 8D graphically illustrate the quantified tumor growth; and,

FIG. 8C and FIG. 8E illustrate representative bioluminescence images of Nalm-6 tumors (FIG. 8B-C) or PC3 tumors (FIG. 8D-E) with (FUS+) or without (FUS−) FUS stimulation;

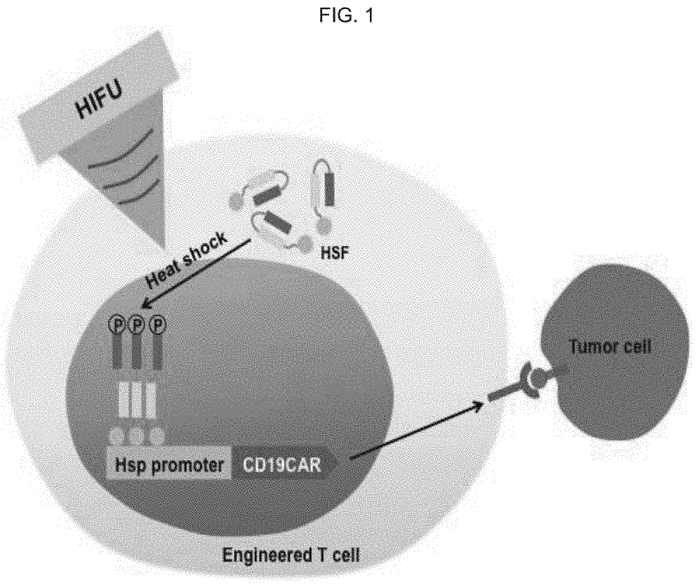

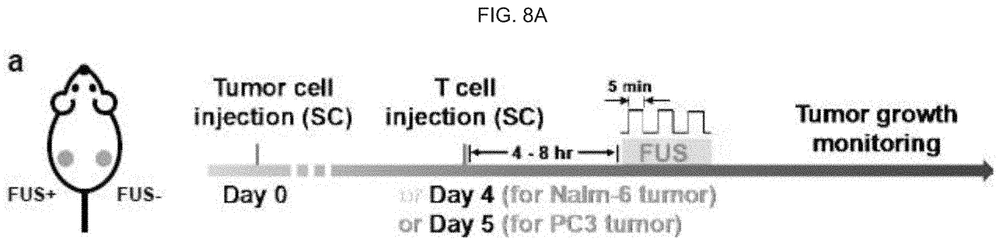

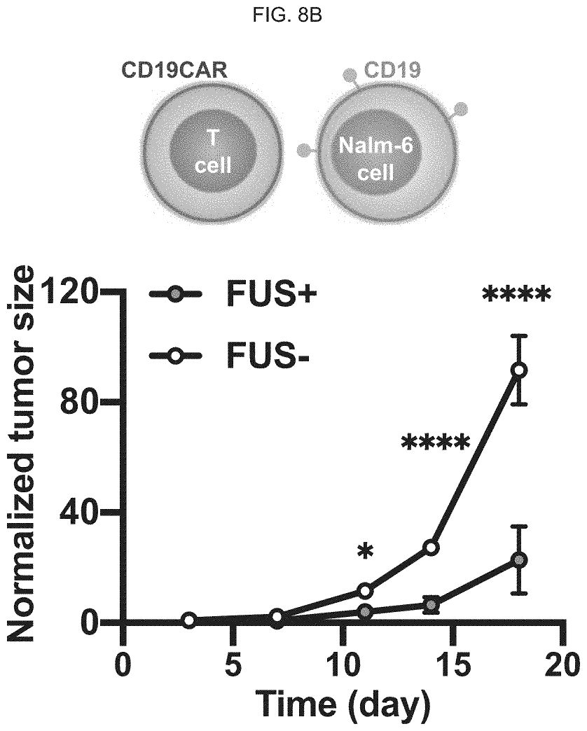

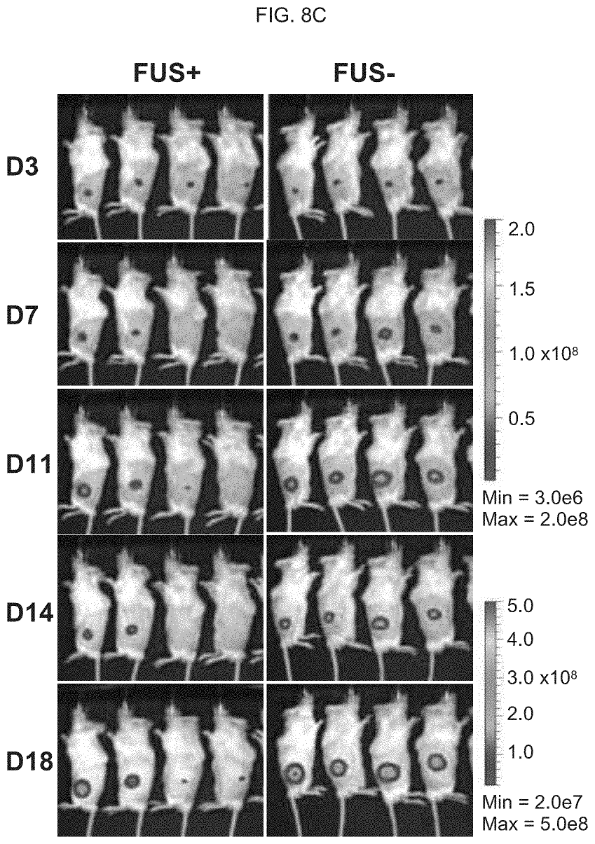

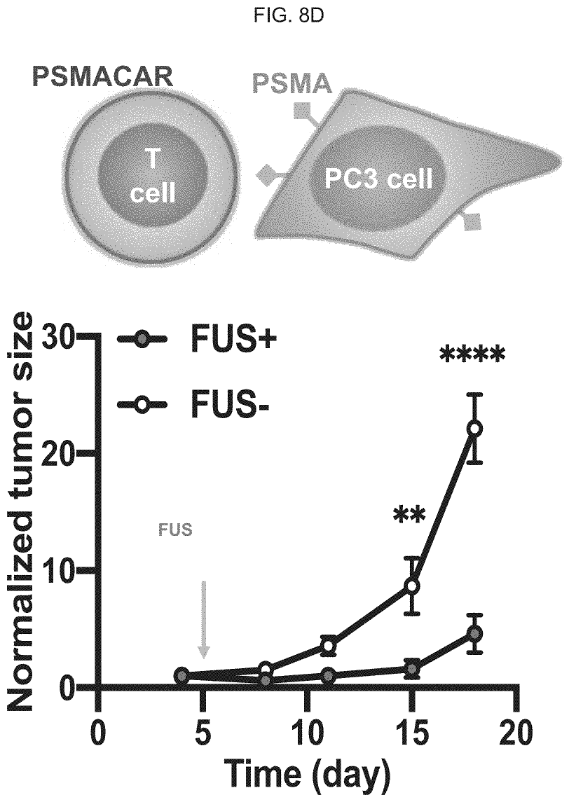

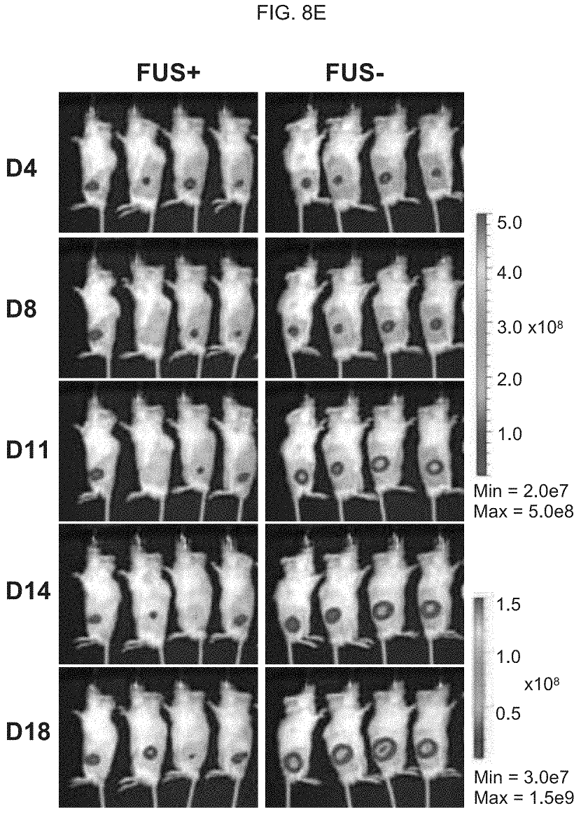

For FIG. 8B and FIG. 8D tumor size was quantified using the integrated Fluc luminescence intensity of the tumor region and normalized to that of the same tumor on the first measurement: in FIG. 8B, *P=2.7×10⁻² at 11, ****P=4.52×10⁻⁶ at 14, ****P=5.12×10⁻¹² at 18; n FIG. 8D, **P=2.6×10⁻³ at 15, ****P=3.31×10⁻⁷ at 18. N=4 mice. Error bar: SEM.

Like reference symbols in the various drawings indicate like elements.

### DETAILED DESCRIPTION

In alternative embodiments, provided are compositions, including products of manufacture and kits, and methods, for remotely-controlled and non-invasive manipulation of physiologic or genetic processes and/or protein expression in live cells in vivo or ex vivo, for example, immune cells such as T cells, for example, the controlled expression of recombinant nucleic acids or proteins such as for example, chimeric T cell or NK receptors, chimeric immunoreceptors or chimeric antigen receptors (CARs), for the manipulation of physiologic processes in the cell or for the correction of pathological processes (for example, non-specific targeting of the CAR-T cells against normal/nonmalignant tissues) and/or for control of therapeutic outcomes, for example, engineered T cells expressing CARs targeting specific cancers cells and killing them.

In alternative embodiments, provided are compositions and methods for the manipulation or correction of pathological processes, for example, for eradicating tumors and cancers in human subjects, without limitation in penetration depth of an inducible signal, that comprise use of ultrasound stimulation. In alternative embodiment, provided are compositions and methods for inducing expression of nucleic acids, for example, genes, in immune cells such as T cells, monocytes/macrophages, dendritic cells, natural killer cells and the like. In alternative embodiment, provided are compositions and methods for stimulating or inhibiting ligand-receptor interactions, including any surface molecular interaction, including but not limiting to inhibitory CTLA-4 and apoptotic Fas.

In alternative embodiments, provided are compositions and methods for the treatment, amelioration, prevention or eradication of a pathologic process or a pathology, a disease, an abnormal tissue, or an infection, for example, bacterial or viral infections, with a specific cell surface marker. In alternative embodiment, provided are compositions and methods for the controlled production of RNAs (including microRNA, long non-coding RNAs), and for the epigenetic and genetic modulation of molecules for the treatment, amelioration, prevention or eradication of a pathologic process, a disease, an abnormal tissue, or an infection.

In alternative embodiments, provided are engineered cells, for example, human cells, for example, immune cells, for example, T cells, capable of inducibly expressing a recombinant protein such as a chimeric antigen T cell or NK receptor (CAR), by operatively linking a gene of interest, i.e., a gene to be turned on and activated by heat or ultrasound (for example, a gene expressing a CAR), to a heat shock promoter such as a human heat shock protein 70B (Hsp) promoter that can be activated by heat shock at about 43° C.

In alternative embodiments, compositions and methods as provided herein can remotely and locally activate engineered chimeric antigen receptor (CAR) T cells by integrating heat-sensitive genetic transducing modules (GTMs) and focused ultrasound for cancer immunotherapy purposes. In immunotherapy, the expression of engineered CAR on the cell surface enables T cells to recognize specific antigens on the target cell. This triggers T cell activation can eventually lead to the elimination of target cells. Clinical trials involving anti-CD19 CAR T cells against B-cell malignancies have shown promising results, demonstrating the therapeutic effects of CAR T cells in cancer treatment. In alternative embodiments, compositions and methods as provided herein address the problem that occurs upon perfusion of constitutively activated CAR T cells into patients, which may have lethal consequences due to the induced cytokine storm and ‘on-target, off tumor’ toxicity, by controlling the timing and location of the activation of the perfused CAR T cells.

In alternative embodiments, high-intensity focused ultrasound (HIFU) and/or focused ultrasound (FUS) is applied to control GTM and transgene activation via a heat-inducible promoter, for example, a heat shock promoter.

In alternative embodiments, compositions and methods as provided herein, by integrating HIFU/FUS and heat-sensitive GTMs, can remotely and non-invasively activate any cell in vivo, including immune cells such as T cells, for example, CAR T cells, with precise spatial and temporal control. In alternative embodiments, the designed GTMs comprise genes or coding sequences of interest (for example, CAR encoding nucleic acids) driven by a heat shock protein, for example, the human heat shock protein 70B (Hsp) promoter that can be activated by heat shock at 43° C. In alternative embodiments, HIFU is applied to generate local heating around cells carrying the GTMs and turn on gene expression.

In alternative embodiments, components of embodiments as provided herein comprise:
Genetic transducing modules (GTMs): the genes of interest driven by a heat shock promoter, which can lead to the expression of genes of interest and thus the production of the therapeutic molecules (for example, anti-CD19 CAR) upon ultrasound heating.
High-intensity focused ultrasound (HIFU) and/or focused ultrasound (FUS) is used for delivering focused mechanical energy to induce local thermal effect to activate GTMs. The heat generated at the target location can activate cells with designed GTMs and induce the desired gene expression.
Cells: any cell can be engineered to incorporate a GTM; for example, human Embryonic Kidney (HEK) 293 cells, immortalized human T cell line Jurkat cells and primary human T cells comprising designed GTMs are used to test the feasibility and efficacy of embodiments as provided herein.

In alternative embodiments: (1) the designed GTMs are introduced into the target cells, and (2) HIFU is applied to generate heat at the region around the target cells. Upon ultrasound induced heat shock, the cells containing the heat-sensitive GTMs are activated and start to express genes of interest. One target gene of interest is CAR, which has therapeutic effects including the triggering of T cell activation and killing of tumor cells.

In alternative embodiments, provided are multiplexed systems comprising use of wireless devices coupling ultrasound transducers (for example, an ultrasound transducer manufactured by Image Guided Therapy, France) such that immunotherapy can be conducted via wireless and remote controls.

In alternative embodiments, the thermomechanical energy of ultrasound can be applied to long-distance therapy, for example, immunotherapy, in deep tissues with high resolutions in space (mm) and time.

In alternative embodiments, provided herein are genetically engineered and remotely controlled cells controllable by short pulsed ultrasound waves at a distance to produce biologically functional molecules and cellular outcomes. In alternative embodiments, immunocells including, but not limited to T cells, monocytes, macrophages and natural killer cells are activated using methods as provided herein. In alternative embodiments, methods as provided herein are used to eradicate tumors and cancers via engineered immunocells in human subjects without limitation in penetration depth.

In alternative embodiments, methods as provided herein are used to remotely control other stimulatory or inhibitory ligand-receptor interactions, as well as any surface molecular interaction, including but not limiting to inhibitory Programmed cell death protein 1 (PD-1), CTLA-4 (cytotoxic T-lymphocyte-associated protein 4) and apoptotic Fas (or apoptosis antigen 1 (APO-1 or APT)).

In alternative embodiments, methods as provided herein are used in the eradication of other diseases or abnormal tissues with specific surface markers, for example, an inducible desmoglein (including for example any desmosomal cadherin such as protein DSG1, DSG2, DSG3, and DSG4) can be utilized to generate CAR immune cells (for example, CAR T cells) to target the surface B cell receptor (BCR) on autoimmune B cells and induce the lysis of these pathogenic B cells for the treatment of autoimmune diseases (see for example, C. R. Maldini, et al., CART cells for infection, autoimmunity and allotransplantation. Nature Reviews Immunology (2018) vol 18, 605-616). In alternative embodiments, methods as provided herein are used to treat or eradicate bacterial or viral infections, e.g. inducible CDL4ζ CAR can be engineered to target HIV infected cells via the surface HIV envelop protein (Env) (see also Maldini, et al.).

In alternative embodiments, methods as provided herein are used are used for the controlled production of RNAs (including microRNA, long non-coding RNAs), epigenetic and genetic modulation molecules for the treatment of diseases.

The extension of the concept to the connection of wearable wireless devices coupling ultrasound transducers such that remote-controlled cell activations can be conducted via wireless and remote controls.

### Products of Manufacture and Kits

Provided are products of manufacture and kits for practicing methods as provided herein; and optionally, products of manufacture and kits can further comprise instructions for practicing methods as provided herein. In alternative embodiments kits as provided herein comprise a recombinantly engineered immune cell such as a T cell, a primary T cell, a B cell, a monocyte, a macrophage, a dendritic cell or a natural killer cell, as provided herein and used to practice methods as provided herein.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Acoustic Thermogenetics for Remote-Controlled Gene Expression and T Cell Activation

This example demonstrates that methods and compositions as provided herein using the exemplary embodiment comprising a use of heat-sensitive genetic transducing modules (GTMs) comprising heat shock protein promoters operatively linked to a gene of interest, for example, a nucleic acid encoding a therapeutic protein, for example, a CAR, are effective and can be used to treat cancer and tumors.

HEK Cells Transfected with the GTMs can be Activated by Heat Shock

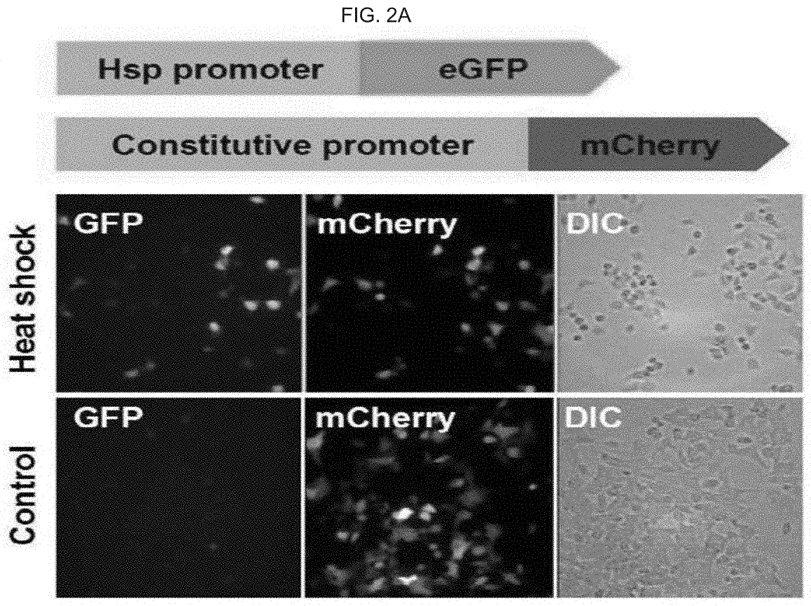

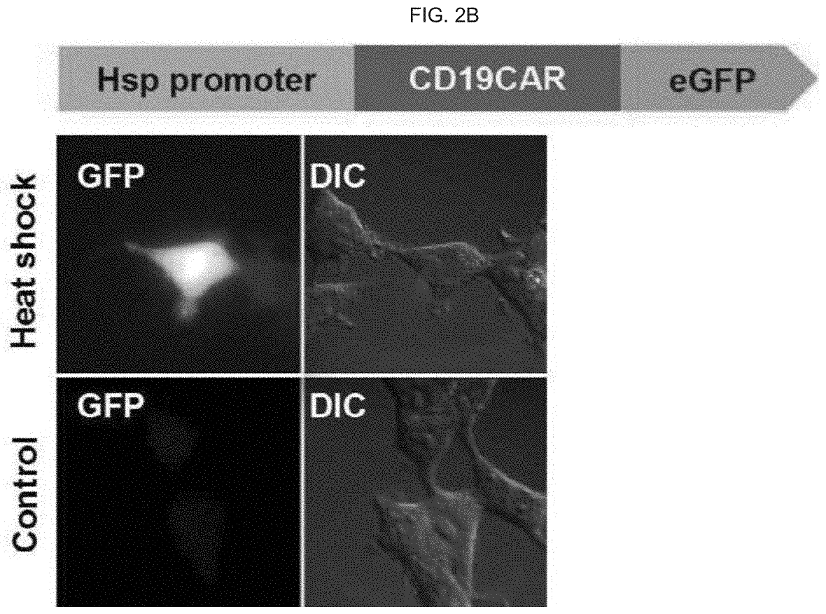

HEK cells were co-transfected with the heat-sensitive GTM (Hsp promoter driving the reporter eGFP (e-green fluorescent protein (GFP))) and a constitutively expressed mCherry. The heat shock stimulation was applied by incubating cells in a 43° C. incubator for 60 min and then transferring to a 37° C. incubator for another 8 hr to 24 hr, while the control cells were maintained at 37° C. during the whole period. The heat shock caused a clear induction of eGFP expression in HEK cells bearing the heat-sensitive GTM (FIG. 2a). Similarly, we tested another heat-sensitive GTM composed of eGFP tagged anti-CD19 CAR driven by the Hsp promoter with the same settings. Again, heat shock successfully induced eGFP expression in HEK cells transfected with this GTM (FIG. 2b). These results demonstrate the feasibility of activating target gene expression by heat shock in HEK cells.

### Jurkat Cells Carrying the GTMs can Express Genes of Interest Upon Heat Shock Activation

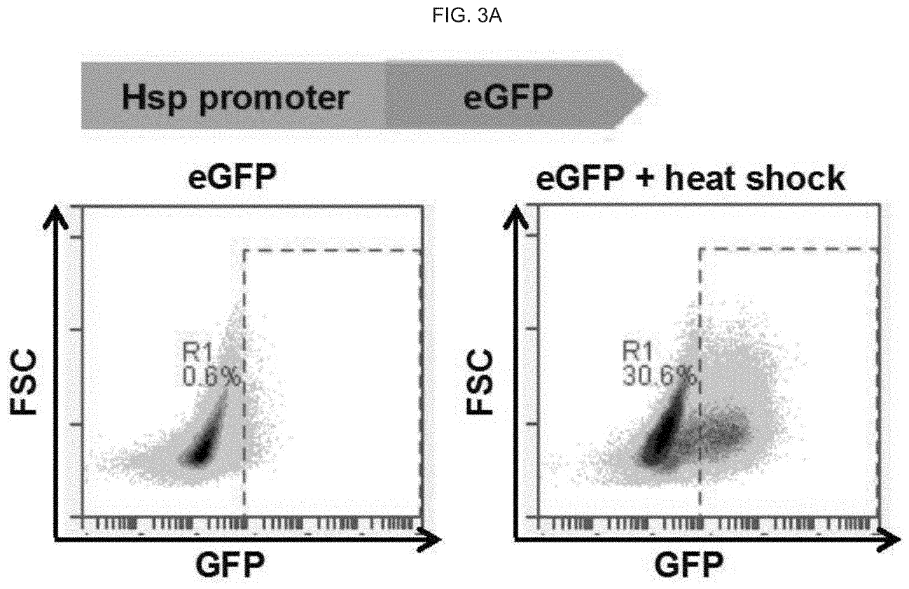

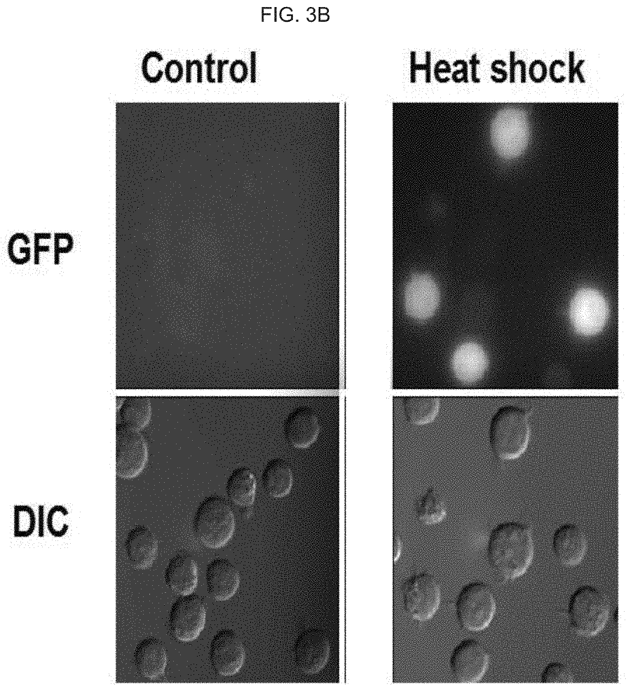

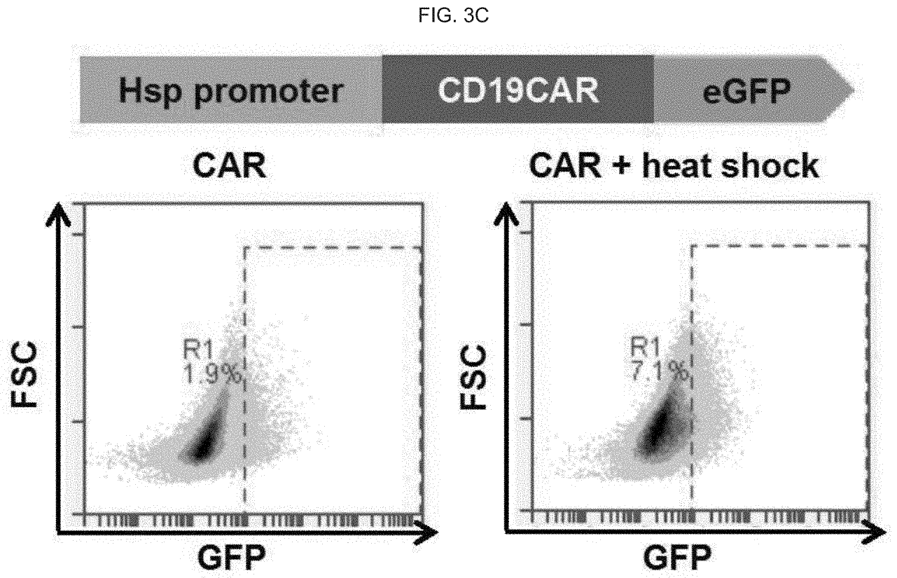

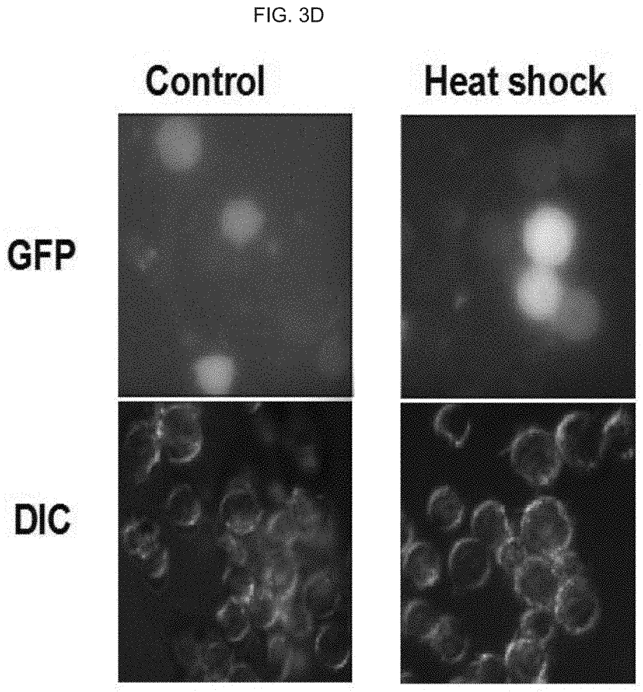

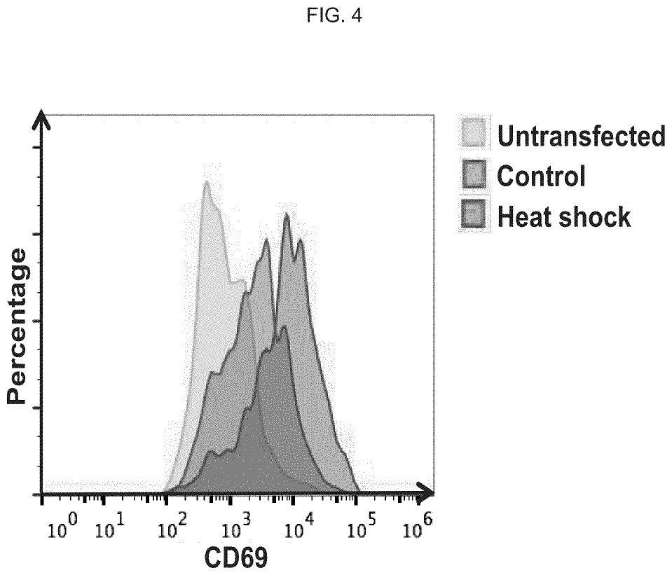

To further test the heat-induced gene activation in T cells, we delivered the designed GTMs into Jurkat cells, an immortalized human T cell line, by either lentiviral infection (FIG. 3) or electroporation (FIG. 4). The engineered Jurkat cells were then heat shocked by incubating in a 43° C. incubator for 60 min, with the control group kept at 37° C. all the time.

For Jurkat cells transfected with the GTM containing the Hsp promoter driven eGFP, the percentage of the cells expressing eGFP increased from 0.8% to 30.6% 13 hr after heat shock as compared to the control (FIG. 3a). Similarly, the GTM containing the eGFP-tagged anti-CD19 CAR driven by the Hsp promoter was also introduced to Jurkat cells. A 2.5-fold increase (from 1.9% to 7.1%) in eGFP expression level was observed in the heat shocked group comparing to the control (FIG. 3b-c). These results indicate that Jurka T cells containing the heat-sensitive GTMs can be induced to express the genes of interest upon heat shock.

### The Expressed Anti-CD19 CAR Induced by Heat Shock is Capable of Triggering Jurkat T Cell Activation

CD69 is a cell surface marker that is increasingly expressed on T cell surface after its activation. To examine the function of the heat-induced anti-CD19 CAR, the Jurkat cells carrying the Hsp promoter driven CD19CAR GTM 12 hr after heat shock were mixed with Toledo cells (B cells expressing CD19 antigen). Upon mixing, the anti-CD19 CAR expressed on the surface of the heat shocked Jurkat cells would interact with the CD19 antigen expressed on the surface of Toledo cells, triggering Jurkat cell activation and the increased expression of CD69. Indeed, the CD69 expression level 24 hr after mixing increased in the heat shocked Jurkat cells as compared to the control groups, either Jurkats hosting GTMs kept at 37° C. during the whole period of experiment (control) or Jurkats not transfected with GTMs but mixed with Toledo cells (untransfected) (FIG. 4). These results suggest that the expressed anti-CD19 CAR induced by heat shock is biologically functional in activating the signaling cascades of Jurkats upon the engagement with target tumor cells.

### The Heat-Sensitive GTMs can be Induced in Primary Human T Cells

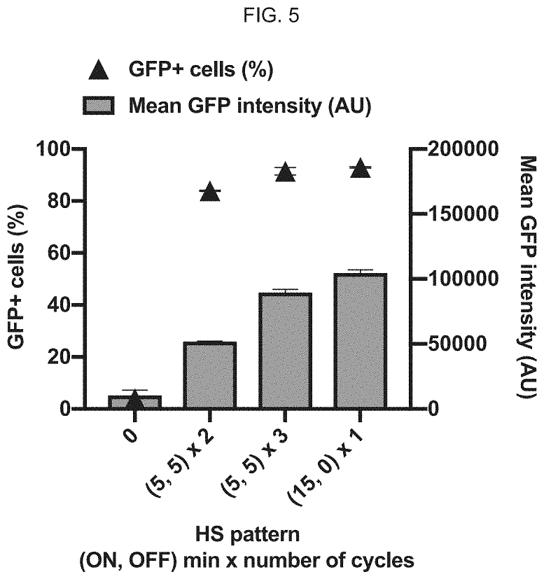

Since Jurkat cells lack killing capacity, we will employ primary human T cells purified from peripheral blood mononuclear cells (PBMCs) for examining the therapeutic effects of the heat-inducible CAR. As a first step, we tested the Hsp promoter mediated gene activation in response to heat shock stimulation in primary human T cells. The heat-sensitive GTM (Hsp promoter-driven eGFP with constitutive mCherry) was introduced into T cells by lentiviral infection. Various patterns of heat shock were then applied as indicated below. More than 80% of the engineered T cells were induced to express eGFP with all the three heat shock patterns tested as compared to a 4% basal activation in the cells without heat shock (FIG. 5), indicating that the heat-sensitive GTM can be activated by short pulsed heat in primary human T cells.

### Heat Induced CAR Expression Enabled T Cells Killing Against Target Tumor

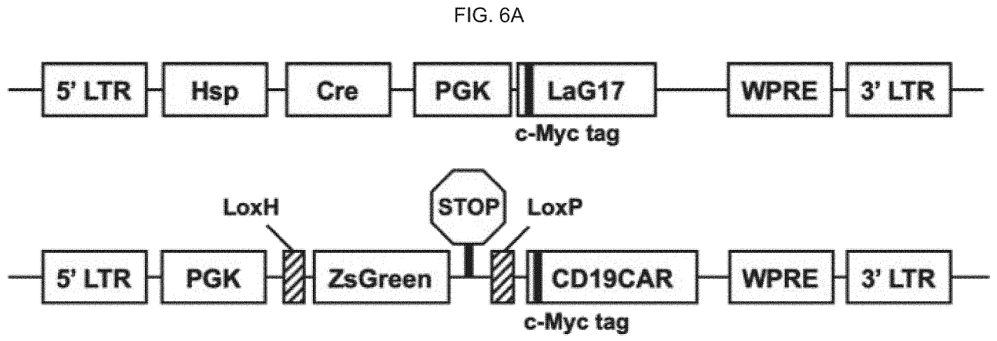

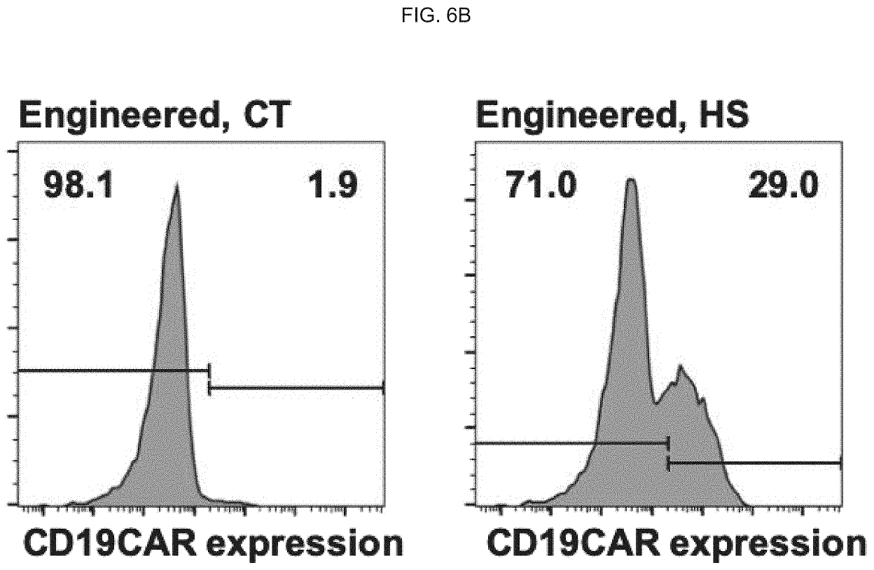

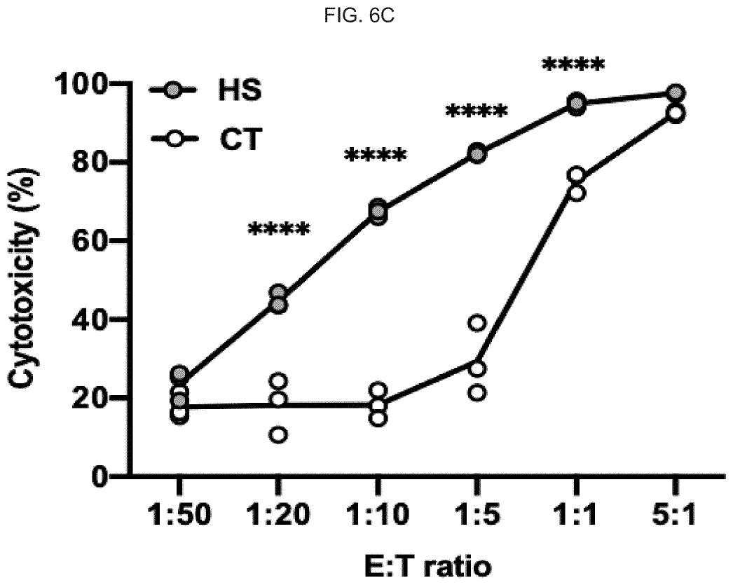

To assess the heat-inducible cytotoxicity of primary human T cells, we lentivirally transduced T cells with heat-sensitive GTM containing a Hsp promoter-driven Cre recombinase with a constitutive c-Myc-tagged LaG17 (GFP nanobody) for sorting purposes, and a lox-flanked stop cassette with the lox-stop-lox sequence between a constitutive PGK promoter and the CAR (FIG. 6a). When cells receive heat stimulation, the Cre recombinase will be produced to excise the lox-flanked stop sequence, resulting in constitutive expression of CAR. Indeed, we observed that 29% of the T cells after heat shock (43° C., 15 min) were induced to express CAR, while the control cells without heat shock showed minimal background of 1.9% (FIG. 6b). The cells were then mixed with firefly luciferase positive target tumor cells at different effector-to-target (E:T) ratios. The cytotoxicity of the T cells was assessed 24 hrs after co-culture. Our results show that T cells stimulated with heat shock possessed stronger cytotoxicity against tumor cells at various E:T ratios including 1:20, 1:10, 1:5 and 1:1 (FIG. 6c).

### MM-Guided FUS System Activates Heat-Sensitive GTMs In Vivo

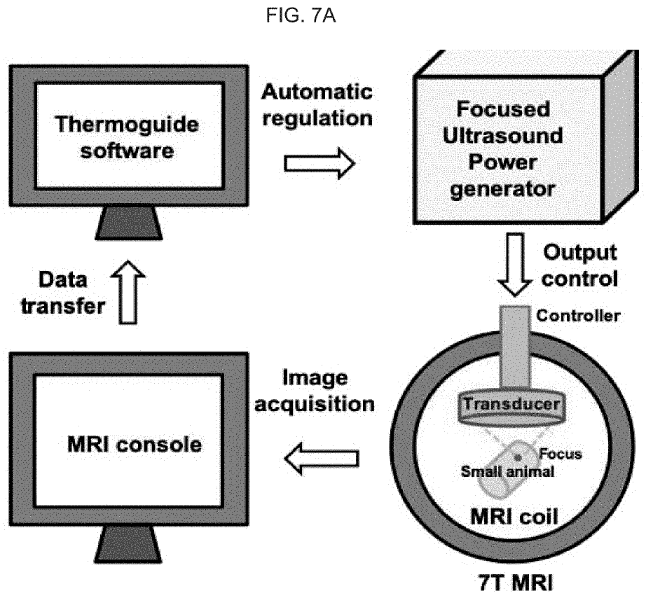

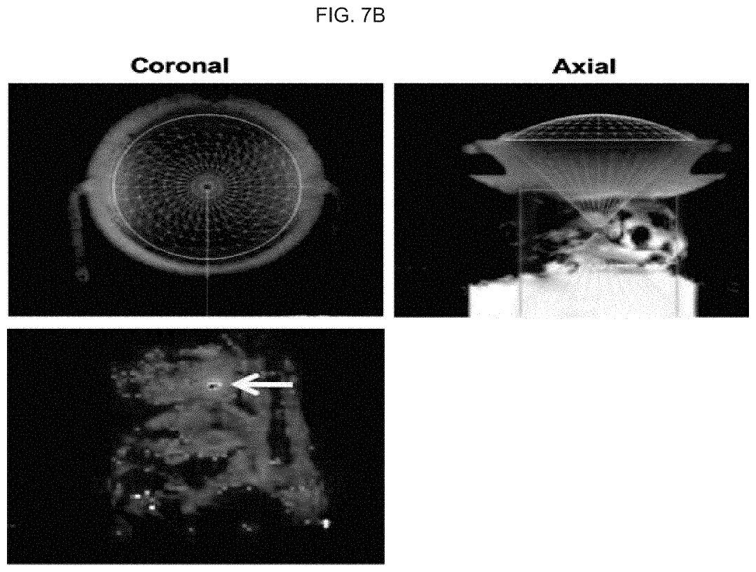

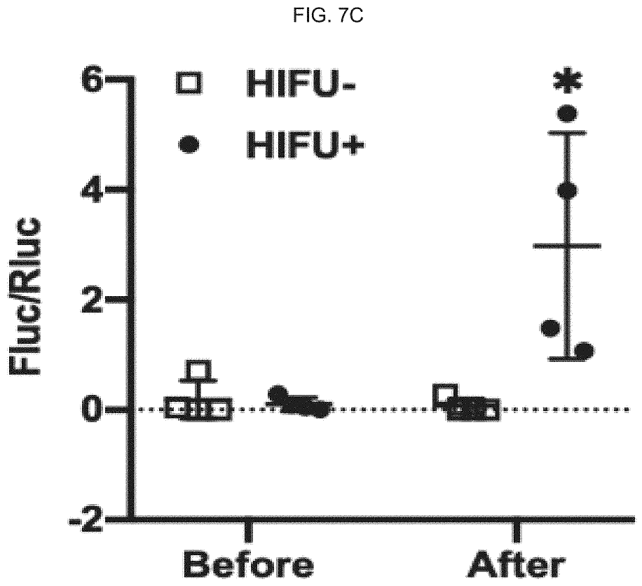

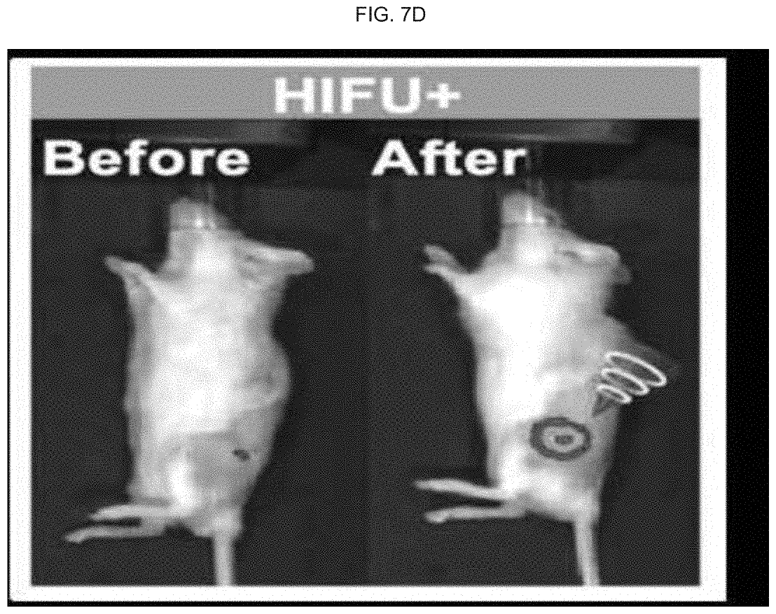

An Mill-guided Focused Ultrasound (FUS) system composed of a 1.5 MHz 8-element annular array transducer, a 16-channel broadband radio-frequency (RF) generator, a piezo motor-based X-Y positioning stage, and a degazing and water circulation system were purchased from IMAGE GUIDED THERAPY™ (France) and installed. MR images acquired using a Bruker 7T™ MRI were transferred to THERMOGUIDE™ software (Image Guided Therapy, France) to generate real-time phase images and temperature maps, allowing automated temperature control at the focal spot via PID controller (FIG. 7a-b).

We further evaluated induced gene activation in vivo by MRI-guided FUS. We engineered a Nalm-6 cell line containing a heat-sensitive GTM of Hsp-driven firefly luciferase (Flue) with constitutive Renilla luciferase (Rluc) serving as an internal control. The ratio of Fluc and Rluc intensities indicates gene activation level. We then subcutaneously injected the engineered Nalm-6 cells to the hindlimb of NSG (NOD scid gamma mouse, Jackson Laboratories) mice, and applied two cycles of “5 min ON, 5 min OFF” FUS stimulation locally at 43° C. We observed that FUS stimulation induced significant gene activation (Fluc/Rluc) in vivo through heat-sensitive GTMs (FIG. 7c-d). These results provide the evidence that MM temperature imaging and FUS can be integrated to remotely and non-invasively control gene activities in vivo with high spatiotemporal resolutions.

FUS Activates T Cells Engineered with Heat-Sensitive GTMs Containing CAR to Suppress Target Tumor Growth In Vivo

We then examined the functionality of the ultrasound-controllable CD19CAR T cells in tumor mouse models. 5×10⁵ Fluc+ Nalm-6 tumor cells were subcutaneously injected to develop tumors in NSG (NOD scid gamma mouse, Jackson Laboratories) mice. After 4 days, primary T cells engineered with or without heat-sensitive GTMs containing Cre and lox-stop CAR (FIG. 6a) were locally injected at the tumor sites and exposed to FUS stimulation (FIG. 8a). Tumor aggressiveness was monitored by bioluminescence imaging (BLI) measuring the integrated luminescence signal of the tumor cells.

FIG. 8A-E illustrates the focused ultrasound (FUS)-controllable tumor suppression by the engineered CAR T cells in vivo: FIG. 8A schematically illustrates the timeline (right image) of the in vivo experiment using NSG (NOD scid gamma mouse, Jackson Laboratories) mouse bearing matched bilateral tumors (left image) as the animal model; the tumor on the left flank received focused ultrasound (FUS) stimulation (FUS+) and the one on the right received no FUS (FUS−) following injection of engineered CART cells; FIG. 8B and FIG. 8D graphically illustrate the quantified tumor growth; and, FIG. 8C and FIG. 8E illustrate representative bioluminescence images of Nalm-6 tumors (FIG. 8B-C) or PC3 tumors (FIG. 8D-E) with (FUS+) or without (FUS−) FUS stimulation;

For FIG. 8B and FIG. 8D tumor size was quantified using the integrated Fluc luminescence intensity of the tumor region and normalized to that of the same tumor on the first measurement: in FIG. 8B, *P=2.7×10⁻² at 11, ****P=4.52×10⁻⁶ at 14, ****P=5.12×10⁻¹² at 18; n FIG. 8D, **P=2.6×10⁻³ at 15, ****P=3.31×10⁻⁷ at 18. N=4 mice. Error bar: SEM.

The results indicate that the FUS-activated CAR T cells can significantly suppress the growth of target tumors in vivo comparing to non-engineered or engineered T cells not exposed to FUS.

A number of embodiments of the invention have been described. Nevertheless, it can be understood that various modifications may be made without departing from the spirit and scope of the invention. Accordingly, other embodiments are within the scope of the following claims.

1. A method for remotely-controlling and non-invasively manipulating expression of an exogenous nucleic acid in a cell, or an immune cell,
the method comprising:
(a) inserting or expressing in a cell, an immune cell or a plurality of cells or immune cells, a thermo-responsive nucleic acid, wherein the thermo-responsive nucleic acid comprises the exogenous nucleic acid operatively linked to a thermo-responsive promoter or a mammalian or a human promoter or transcriptional activator activated by increased temperature, to form a Gene Transducing Module (GTM),
(b) stimulating or exposing the cell to a heat or a heat source sufficient to cause the thermo-responsive promoter to be activated, thereby causing the thermoresponse protein to be expressed in the cell.

2. The method of claim 1, wherein the expressing of the thermoresponse protein in the cell adds a function to the cell, or immune cell, or manipulates a physiologic and/or a genetic process in the cell, or immune cell.

3. The method of claim 1, wherein the cell is a human cell or a mammalian cell, or is a recombinantly engineered cell engineered to contain or comprise the Gene Transducing Module (GTM), transplanted or inserted into a tissue, an organ, an organism or an individual, or is a non-human transgenic animal genetically engineered to contain and express the Gene Transducing Module (GTM) or vector.

4. The method of claim 1, wherein the cell is exposed to a high-intensity focused ultrasound (HIFU) and/or focused ultrasound (FUS),
thereby generating sufficient heat in the cell to cause the thermo-responsive promoter to be activated,
thereby causing the thermoresponse protein to be expressed in the cell.

5. The method of claim 1, wherein the cell is inside the body of an animal or a human in need thereof, and the HIFU and/or FUS is focused on or approximate to a tumor or a dysplastic or dysfunctional tissue.

6. The method of claim 1, wherein the method is used for the manipulation or correction of a pathological process.

7-9. (canceled)

10. The method of claim 1, further comprising modifying or adding a target capability or a function to the cell, or immune cell.

11. The method of claim 1, wherein the immune cell is a T cell, a primary T cell, a B cell, a monocyte, a macrophage, a dendritic cell or a natural killer cell.

12. The method of claim 1, wherein the exogenous nucleic acid is contained in a vector or expression cassette.

13. The method of claim 1, wherein the exogenous nucleic acid comprises a nucleic acid encoding or expressing a protein.

14. The method of claim 13, wherein the protein is a therapeutic protein, or a transcriptional or translational regulatory protein, or a receptor, or a recombinant or an artificial T cell or NK receptor (also known as a chimeric T cell or NK receptor, a chimeric immunoreceptor, a chimeric antigen receptor and a CAR), an antibody, a single chain antibody, or a single-domain antibody (also known as sdAb or nanobody) or an antibody fragment consisting of a single monomeric variable antibody domain.

15. The method of claim 1, wherein the thermo-responsive promoter is or comprises a heat shock protein (Hsp) promoter, or a human heat shock protein 70B (Hsp) promoter.

16. The method of claim 2, wherein when the upregulated nucleic acid is a nucleic acid expressing or encoding a CAR, a single chain antibody, or a single-domain antibody (also known as sdAb or nanobody) or an antibody fragment consisting of a single monomeric variable antibody domain, thereby adding a new specificity, function or target cell to a cell, an immune cell or a T cell.

17. The method of claim 4, wherein the cell is heated to between about 40° C. and 48° C.

18. The method of claim 17, wherein the cell is heated to between about 42° C. and 45° C., or to about 43° C.

19. The method of claim 6, wherein the individual is a human or an animal.


SEQUENCE LISTING (1 sequence)

SEQ ID NO: 1 - DNA, 426 bp, homo sapien
gtcgaggcgcgtcctcagagccagccgggaggagctagaaccttccccgcgtttctttcagcagccctgagtcagaggcgggctggcctggcatagccgcccagcctctcggctcacggcccgatccgcccgaaccttctcccggggtcagcgccgcgctgcgccgcccggctgactcagcccgggcgggcgggcgggaggctctcgactgggcgggaaggtgcgggaaggttcgcggcggcggggtcggggaggtgcaaaaggatgaaaagcccgtggaagcggagctgagcagatccgagccgggctggcggcagagaaaccgcagggagagcctcactgctgagcgcccctcgacggcggagcggcagcagcctccgtggcctccagcatccgacaagaagctctctagtcgacggtatcgat